(19) United States
(12) Patent Application Publication
(10) Pub. No.: US 2026/0272980 A1
(43) Pub. Date: Sep. 17, 2026

(54) CHIMERIC ANTIGEN RECEPTORS AND THEIR DERIVATIVES AND APPLICATIONS

(71) Applicant: FUDAN UNIVERSITY, Shanghai (CN)

(72) Inventors: Di ZHU, Shanghai (CN); Ke YU, Shanghai (CN); Zhongen WU, Shanghai (CN)

(21) Appl. No.: 19/116,709

(22) PCT Filed: Sep. 28, 2022
(86) PCT No.: PCT/CN2022/122211

§ 371 Date: Mar. 28, 2025

(51) Int. Cl. A61K 35/17; A61K 40/11; A61K 40/31; A61K 40/42; A61P 35/00; C12N 15/86

(52) U.S. Cl. CPC A61K 35/17; A61K 40/11; A61K 40/31; A61K 40/4202; A61P 35/00; C12N 15/86; A61K 2239/21; A61K 2239/49; C12N 2740/16043

(57) ABSTRACT

The embodiments of the present invention provide a chimeric antigen receptor with a sequence of a light chain variable region as shown in SEQ ID NO: 1 and a sequence of a heavy chain variable region as shown in SEQ ID NO: 2, which can have a cytotoxic effect on tumor cells, especially tumor cells expressing Axl. The embodiments of the present invention also provide derivatives of the chimeric antigen receptor and their applications.

### CROSS-REFERENCE TO A PRIOR APPLICATION

This is a National Phase Application filed under 35 U.S.C. 371 as a national stage of PCT/CN2022/122211, filed Sep. 28, 2022, the content of which is hereby incorporated by reference in its entirety.

### SEQUENCE LISTING

The instant application contains a Sequence Listing which has been submitted in XML format via Patent Center and is hereby incorporated by reference in its entirety. Said XML copy, created on Jan. 27, 2026, is named 41056U_Sequence_Listing.xml and is 22,825 bytes in size.

### FIELD OF TECHNOLOGY

The present invention belongs to the field of genetic engineering technology, and particularly relates to chimeric antigen receptors and their derivatives and applications.

### BACKGROUND

Axl is a receptor tyrosine kinase, which is highly expressed in a variety of cancers, such as granulocytic leukemia, pyrethroid leukemia, megakaryocytic leukemia, endometrial cancer, gastric cancer, colon cancer, prostate cancer, thyroid cancer, lung cancer, breast cancer, ovarian cancer, liver cancer, renal cell cancer, glioblastoma, melanoma, osteosarcoma, and is a potential anticancer drug target.

Therefore, it is necessary to provide novel Axl specific chimeric antigen receptors.

### SUMMARY

The present invention provides a chimeric antigen receptor that has a cytotoxic effect on tumor cells, particularly tumor cells expressing Axl.

The chimeric antigen receptor of the present invention comprises Axl specific antibodies, which can be single chain antibodies (scFV), monoclonal antibodies, domain antibodies, Fab fragments, Fd fragments, Fv fragments, F(ab′)2 fragments and their derivatives, or other forms of antibodies.

Preferably, the Axl specific antibody is a humanized antibody. The humanized antibody is a full-length antibody, and the subtype is any one of hIgG1, hIgG2, hIgG3, and hIgG4.

Preferably, the sequence of the light chain variable region is shown in SEQ ID NO: 1; The sequence of the heavy chain variable region is shown in SEQ ID NO: 2.

Preferably, the Axl specific antibody further comprises a linker region located between the light chain variable region and the heavy chain variable region, as shown in SEQ ID NO: 3.

Preferably, the Axl specific antibody is formed by sequentially connecting the light chain variable region, the linker region, and the heavy chain variable region.

Preferably, the chimeric antigen receptor further comprises a hinge region connecting the C-terminus of the heavy chain variable region of the Axl specific antibody, and a transmembrane domain connecting the hinge region.

Preferably, the sequence of the hinge region is derived from at least one of CD8α, CD28, 4-1BB, ICOS, OX40, CD40, CD80, and IgG.

Preferably, the sequence of the transmembrane domain is derived from at least one of CD2, CD27, LFA-1, CD8α, CD28, 4-1BB, ICOS, OX40, CD40, CD80, CD3ζ, and CD3ε.

Preferably, the sequence of the hinge region is shown in SEQ ID NO: 4, and the sequence of the transmembrane domain is shown in SEQ ID NO: 5.

Preferably, the chimeric antigen receptor further comprises an expression driver region and a signal peptide connecting the expression driver region and the light chain variable region of the AXL specific antibody.

The expression driving region of the present invention is capable of labeling and encoding promoters for the expansion and suicide of CAR cells.

Preferably, the sequence of the expression driving region is derived from at least one of EF1α, CMV, PGK, MPSV, MMLV, and SFFV.

Preferably, the sequence of the expression driver region is shown in SEQ ID NO: 6.

Preferably, the sequence of the signal peptide is shown in SEQ ID NO: 7.

Preferably, the sequence of the chimeric antigen receptor is shown in SEQ ID NO: 8.

The present invention also provides a nucleic acid encoding at least a partial fragment of the chimeric antigen receptor, as well as a vector comprising the nucleic acid.

Preferably, the vector can be an adenovirus vector, a lentiviral vector, or a retroviral vector.

The present invention also provides a vector comprising the nucleic acid and a host cell comprising the vector.

Preferably, the host cells can be recombinant microbial host cells or mammalian cells.

Preferably, the host cell is capable of expressing the Axl specific antibody.

The present invention also provides the application of the chimeric antigen receptor in the preparation of tumor related drugs and tumor treatment.

Preferably, the tumor cell lines are with high expression of Axl.

### BRIEF DESCRIPTION OF THE DRAWINGS

FIG. 1. The plasmid map of the shuttle plasmid in an embodiment of the present invention;

FIG. 2. The comparison of flow cytometry results between the transfection control group and the transfection experimental group in an embodiment of the present invention;

FIG. 3. The comparison of mRNA relative expression levels of anti Axl scFv between the transfection control group and the transfection experimental group in an embodiment of the present invention;

FIG. 4. The blank group of LCLC-103H in the embodiment of the present invention and in E:T=4:1. Comparison chart of the changes in LCLC-103H proliferation rate between the control group and the experimental group at different co-culture times under condition 1;

FIG. 5. The comparative trend of MDA-MB-231 proliferation rate under different co-culture times in the blank group, control group corresponding to different E:T, and experimental group corresponding to different E:T of the MDA-MB-231 embodiment of the present invention;

FIG. 6. The proliferation rate trends of the corresponding control group and experimental group of LCLC-103H according to an embodiment of the present invention at different E:T ratios, as well as the comparison of the proliferation rate trends of the corresponding control group and experimental group of H460 at different E:T ratios;

FIG. 7. The proliferation rate trends of the corresponding control group and experimental group of MDA-MB-231 at different E:T, as well as the comparison of the proliferation rate trends of the corresponding control group and experimental group of MDA-MB-453 at different E:T, according to an embodiment of the present invention;

FIG. 8. The comparison of relative expression levels of IFNγ among the blank group, control group, and experimental group corresponding to MV4-11 in the embodiment of the present invention;

FIG. 9. The comparison of relative expression levels of IFNγ among the blank group, control group, and experimental group corresponding to HL60 in the embodiment of the present invention;

FIG. 10. The comparison of relative expression levels of IFNγ among the blank group, control group, and experimental group corresponding to MDA-MB-231 in the embodiment of the present invention;

FIG. 11. A comparative photo of tumor metastasis in lung tissue samples from the PBS group, animal control group, and animal treatment group according to an embodiment of the present invention;

FIG. 12. A comparison of the proportion of lung metastasis areas in each group of lung tissue samples based on the photo shown in FIG. 11;

FIG. 13. A comparison of immunostaining images of in situ tumor tissue samples from the PBS group, animal control group, and animal treatment group according to an embodiment of the present invention;

FIG. 14. A comparison of the proportion of positive areas for CD3 and CD8 in the in situ tumor tissue samples of the PBS group, animal control group, and animal treatment group according to an embodiment of the present invention;

FIG. 15. The comparative expression levels of CD3, CD8A, CD4, IFNG, GZMB, and TNFA in the PBS group, animal control group, and animal treatment group according to an embodiment of the present invention.

### DESCRIPTION OF THE EMBODIMENTS

In order to clarify the purpose, technical solution, and advantages of the embodiments of the present invention, a clear and complete description of the technical solution in the embodiments of the present invention will be provided below. Obviously, the described embodiments are a part of the embodiments of the present invention, not all of them. Based on the embodiments of the present invention, all other embodiments obtained by ordinary skilled persons in the art without creative labor are within the scope of protection of the present invention. Unless otherwise defined, the technical or scientific terms used herein shall have the usual meanings as understood by those skilled in the art to which the present invention belongs. The use of similar words such as “including” in this article means that the elements or objects that appear before the word encompass the elements or objects listed after the word and their equivalents, without excluding other elements or objects.

The technical terms used in the embodiments of the present invention have the same or similar meanings as those commonly understood by those skilled in the art. For the convenience of understanding the present invention, some terms are defined as follows:

In the embodiments of the present invention, “antibody” refers to the antigen binding protein of the immune system, including a complete full-length antibody with an antigen binding region and any fragment thereof retained in the “antigen binding portion” or “antigen binding region”, or a single chain such as a single chain variable fragment (scFv). Antibody “also includes all recombinant forms of antibodies (especially the antibodies described in this article), such as antibodies expressed in prokaryotic cells, unglycosylated antibodies, and antibody fragments and derivatives that bind to antigens.

“CD3ζ” is defined as a protein provided by GenBan accession number BAG36664.1, or an equivalent residue from non-human species such as mice, rodents, monkeys, apes, etc. The “CD3 ζ domain” is defined as the amino acid residues from the cytoplasmic domain of the Zeta chain, which are sufficient to functionally transmit the initial signals required for T cell activation. On the one hand, the cytoplasmic domain of Zeta contains residues 52 to 163 of GenBank accession number BAG36664.1, which functionally point to homologs-equivalent residues from non-human species such as mice, rodents, monkeys, apes, etc.

“CD28” is defined as a protein provided by GenBan accession number NP_006130.1, or an equivalent residue from non-human species such as mice, rodents, monkeys, apes, etc. The “CD28 signaling region” is defined as the amino acid residues from the cytoplasmic domain of CD28, which can transmit the co stimulatory signals required for T cell activation; Its sequence contains residues 180 to 220 of GenBank accession number NP_006130.1, and its functional homology refers to equivalent residues from non-human species such as mice, rodents, monkeys, apes, etc. The “CD28 hinge region” contains residues 114 to 152 of GenBank accession number NP_006130.1, which functionally point to homologs-equivalent residues from non-human species such as mice, rodents, monkeys, apes, etc. The “CD28 transmembrane region” contains residues 153 to 179 of GenBank accession number NP_006130.1, which functionally point to homologs-equivalent residues from non-human species such as mice, rodents, monkeys, apes, etc.

“CD8” is defined as a molecule that is a leukocyte differentiation antigen, a glycoprotein on the surface of some T cells, used to assist T cell receptors (TCRs) in recognizing antigens and participating in the transduction of T cell activation signals, also known as TCR co receptors;

The “CD8 hinge region” is defined as a variable region (V-like region) present in the extracellular parts of both the alpha and beta chains of CD8 molecules, with a hinge region (linker peptide) rich in proline, threonine, and serine residues between the V-like region and the membrane.

“EF1 alpha” is defined as a strong mammalian expression promoter of the elongation factor 1 alpha (EF1A) gene, which can stably drive the constitutive expression of downstream genes in various cells. The EF1 alpha promoter is suitable for expression in stem cells, primary cells, hematopoietic cells, etc., and is weaker than CMV in commonly used cell lines such as HEK293 and tumors.

Axl, also known as Ark, Ufo or Tyro-7, belongs to the receptor tyrosine kinase TAM family. Its extracellular segment is structurally similar to the neural cell adhesion molecule (NCAM), containing two immunoglobulin like regions (Ig) and two fibronectin III (FN3) like regions. The Ig like region is the area that binds to ligands, while the FN III region plays a regulatory role in the binding of Axl protein to its ligands. The intracellular region of Axl protein is a tyrosine kinase like region with kinase activity.

The reagents and consumables involved in the embodiments of the present invention can be purchased through commercial channels, and unless otherwise specified, they are all from Hanheng Biotechnology (Shanghai) Co., Ltd.

The cell culture described in the embodiments of the present invention, unless otherwise specified, is carried out in a carbon dioxide cell culture incubator, with a controlled temperature of 37±0.5 degrees Celsius and a carbon dioxide volume concentration of 5±0.5%.

The qPCR confidence interval in the embodiments of the present invention is 95%. For data involving statistical analysis, each experiment should be repeated at least 3 times, and the experimental results should be analyzed using GraphPad Prism 8.0 software. Two sets of data were compared using a two tailed unpaired t-test to calculate statistical differences, while multiple sets of data were compared using ANOVA analysis of variance to calculate statistical differences. P<0.05 is considered statistically significant, and in the accompanying figure of the instruction manual: *represents P<0.05***Represents P<0.001.

### Example 1: Axl Specific Chimeric Antigen Receptor

This embodiment provides an Axl specific chimeric antigen receptor (abbreviated as Axl CAR) with the sequence shown in SEQ ID NO: 8.

### Example 2: Construction of Shuttle Plasmid

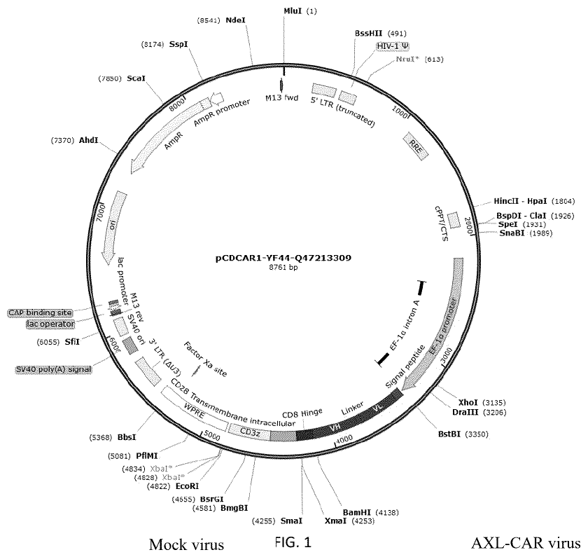

In this example, the nucleotide sequence of Axl CAR from Example 1 was amplified by PCR and digested with restriction enzymes. The recovered digested fragments were inserted into the multi cloning site of the shuttle plasmid to obtain the recombinant shuttle plasmid. Please refer to FIG. 1 for the plasmid map of the shuttle plasmid; The specific operation process is a conventional technical means for technical personnel in this field.

In this example, the DH5 α strain in E. coli DH5 α competent cell suspension (product number MCC0010, Frdbio) was used as the receptor cell. 10 nanograms of the recombinant shuttle plasmid obtained above were added to every 100 microliters of E. coli DH5 α competent cell suspension and mixed. After being placed in an ice bath for 20-30 minutes, the recombinant shuttle plasmid was thermally shaken at 42 degrees Celsius for 90 seconds to enter the cell for transformation. Please refer to the product manual of E. coli DH5 α competent cell suspension for specific operating procedures, which will not be elaborated here.

Add 100 microliters of the transformed cell suspension to 300 microliters of fresh LB free liquid medium, mix well, and shake at 37° C. and 230 rpm for 45-60 minutes. Then, spread evenly in a 10 centimeter culture dish pre coated with 20 milliliters of fresh LB free solid medium. After uniform coating, the culture dish was inverted and incubated at 37 degrees Celsius for 12-16 hours until obvious and non overlapping single colonies appeared. After color development, white colonies were selected and cultured in fresh LB free liquid as the bacterial solution for PCR sequencing identification. The reaction system consisted of 2 microliters of bacterial solution, 5 microliters of 2×Hieff PCR Master MIX, 2 microliters of ddH₂O, 0.5 microliters of upstream primer, and 0.5 microliters of downstream primer. The sequencing results confirmed that the sequencing results were consistent with the target sequence.

The upstream primer sequence is 5 ‘-GTGTGACCCTGACTGTGGAC-3’, and the downstream primer sequence is 5 ‘-AGTAGTTCGAAGACGCGGTG-3’.

The recombinant shuttle plasmid bacterial liquid was amplified and purified by plasmid extraction kit (Tiangen, DP117), and the purified recombinant shuttle plasmid was used for transfection into cells.

### Example 3: Slow Virus Packaging

This embodiment packages the purified recombinant shuttle plasmid from Example 2 as a lentivirus, and the specific packaging method is as follows:

293T cells were cultured in fresh complete medium DMEM (Thermo, item number 11965118) containing 10% fetal bovine serum until the confluence degree was 70-80%, and then resuspended to obtain a cell suspension (cell number 5×10⁶, volume 20 mL). The mixture of 10 μg pSPAX2 plasmid, 5 μg pMD2G plasmid, 10 μg purified recombinant shuttle plasmid package, and 75 μL transfection reagent (Hanbio Biotechnology, item number HB-TRCF-1000) was incubated at room temperature for 15 minutes, and then slowly added dropwise to the above cell suspension for transfection. 16 hours after transfection, the culture medium was replaced. 48 hours after transfection, the culture was centrifuged at 2000 g centrifugal force and 4 degrees Celsius for 10 minutes. The virus supernatant was collected and filtered using a sterile filter to remove cell debris and impurities. Then, the supernatant was centrifuged at 82700 g centrifugal force and 4 degrees Celsius for 120 minutes to obtain a lentivirus precipitate; Resuspend the lentivirus precipitate using 200 microliters of the aforementioned DMEM complete culture medium to obtain a lentivirus resuspension.

Take 10 microliters of lentivirus suspension and add it to a 96 well plate pre inoculated with Hela cells for 24 hours. After culturing, perform microscopic examination and observe that the culture medium is clear and transparent, with no obvious particles in the intercellular space and no bacterial or fungal contamination.

After incubating 10 microliters of lentivirus suspension in a 96° C. water bath for 15 minutes, PCR reaction was performed. No obvious bands were observed in the PCR gel, indicating that the lentivirus was not contaminated with mycoplasma.

The specific steps for titer detection of lentivirus resuspended solution are as follows:

On the first day, 293T cells with good proliferation status were digested and counted, diluted to 1×10⁵/mL, and added to a 96 well plate for 48 hours of cell culture.

The next day: Prepare 6 1.5 mL EP tubes, add 10 μL of lentivirus resuspension to the first EP tube, and then perform a 3-fold gradient dilution to obtain 6 dilutions of lentivirus resuspension.

On the third day, if there are wells that need to be screened with puromycin, first aspirate 100 mL of virus containing culture medium and add 100 μL of 10% FBS complete culture medium containing 1.5 μg/mL puromycin.

On the fifth day, observe the results under a fluorescence microscope. Six hours before observing the results, replace with fresh 10% FBS complete culture medium. Suck out 80 μL of culture medium from the well, then add 80 μL of fresh 10% FBS complete culture medium and incubate in a 37° C., 5% CO₂ incubator. After 6 hours, observe the results under a fluorescence microscope and calculate the virus titer for wells with a fluorescence percentage of 10-50%.

$$Titer ( TU / mL ) = number of cells \times percentage of positive clones \times MOI ( 1 ) \times dilution factor of virus \times {10}^{3} TU / mL$$

The virus titer was calculated to be 1×10⁸ TU/ml.

### Example 4: Construction of Axl-CAR-T Cells

This example infects human peripheral blood mononuclear cells (PBMCs) with the lentivirus suspension obtained in Example 3 to construct Axl-CAR-T cells.

The specific construction process is as follows:

Take 10 milliliters of blood samples from healthy volunteers, dilute them with 20 milliliters of PBS, and slowly add them to 10 milliliters of Ficoll Paque PLUS reagent (GE healthcare). Centrifuge at room temperature for 30 minutes under 800 g centrifugal force to remove the serum layer. Draw the white flocculent cell layer in the middle into a centrifuge tube and add 30 mL of PBS diluted cell solution. Then centrifuge at room temperature for 15 minutes under 400 g centrifugal force to discard the supernatant. Resuspend the precipitate obtained with 1 mL of PBS, filter it through a 70 μm filter membrane, and centrifuge at 1500 rpm for 5 minutes at room temperature. Discard the supernatant and resuspend the cells in RPMI complete medium with 200 U/mL rhIL-2 (Biolegend) to 1×10⁶ cells/mL. After 2 hours of cultivation, the human T cell activation/proliferation kit (Milton Biotec GmbH) was used for stimulation, followed by 4-5 days of cultivation and passaging every two days to obtain the T cells to be transfected.

Collect the culture products containing T cells to be transfected obtained above, centrifuge at room temperature for 5 minutes at 1500 rpm, and discard the supernatant. The obtained precipitate was resuspended in EasySep Buffer and transferred to a 5 mL round bottomed test tube, which was then placed in a magnet for 5 minutes. Tilt the magnet and test tube together, pour out and collect the cell fluid, then centrifuge at room temperature for 5 minutes at 1500 rpm, discard the supernatant, and resuspend the cells in RPMI medium to 1×10⁶ cells/mL to form a suspension of cells to be transfected.

According to the virus titer of 1×10⁸ TU/ml and the selected MOI (multiplicity of infection), calculate the amount of lentivirus used. Take 250 microliters of cell suspension to be transfected and add them to the transfection system to form the following transfection control group and transfection experimental group:

Transfection control group: Add 0.5 μL of polyamine (final concentration 5 μg/mL) and 0.5 μL of IL-2 (final concentration 5 μg/mL), control MOI=5. The difference between the added control virus suspension and the lentivirus suspension in Example 3 is that the control shuttle plasmid is packaged as a control lentivirus according to the method described in Example 3, and the control shuttle plasmid is obtained by following the steps for preparing recombinant shuttle plasmids in Example 2, except that GFP protein sequence is used instead of Axl CAR sequence.

Transfection experimental group: The difference from the transfection control group is that the virus suspension added is the lentivirus suspension obtained in Example 3.

Centrifuge the transfection control group and transfection experimental group at 200 g centrifugal force and room temperature for 1 hour, respectively, and culture for 6 hours. Then add RPMI complete culture medium containing 200 U/mL rhIL-2 to each group for replenishment to a sample volume of 500 microliters per group, and continue to culture for 48 hours.

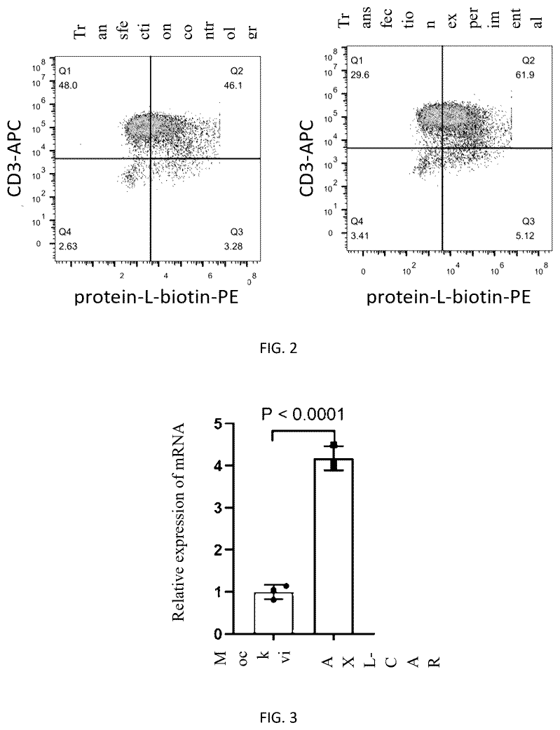

In this example, the cells obtained from each group were cultured for 2 days and stained with biotinylated protein L, streptavidin PE, and anti-CD3-APC. Then, flow cytometry was used for detection, and the flow cytometry results of the transfection control group and transfection experimental group were compared as shown in FIG. 2. The staining step and flow cytometry detection step are conventional techniques used by technicians in this field. Referring to FIG. 2, the comparison between the transfection control group and the transfection experimental group shows that after transfection with the lentivirus of Example 3, T cells successfully expressed Axl CAR.

In this example, the cells obtained from each group were cultured for 2 days and lysed. Total RNA was extracted and reverse transcription was performed. The obtained cDNA was used as a template, and qPCR was performed using GAPDH as an internal reference gene. The relative expression levels of anti Axl scFv mRNA in the transfection control group and transfection experimental group were compared as shown in FIG. 3. The steps of total RNA extraction, reverse transcription, and qPCR are conventional techniques used by those skilled in the art. Referring to FIG. 3, compared with the transfection control group, the relative expression level of anti Axl scFv mRNA in the transfection experimental group was significantly increased by about 3 times.

Perform qPCR using Takara PrimeScript® The RT Master Mix reagent is used to prepare the reaction system, and the primer pair sequence for the target gene is as follows:

AXL-humab1-F:
5′-GGTGAGAGCAAGTTCA-3′
and
AXL-humab1-R:
5′-CTCTAAACTTTGCGGGAG-3′;
CD3-F:
5′-TGCCCTTATCAGTTGGCGT-3′
and
CD3-R:
5′-TTCCTGGGGTAGCGACA-3′;
CD8-F:
5′-TACTGCAACCAGGAACCG-3′
and
CD8-R:
5′-AGTAATCTTTCCCACCCCGC-3′;
CD4-F:
5′-CCAGGGCCCTTC-3′
and
CD4-R:
5′-TTCTTCCCTGAGTGGCTGC-3′;
IFNG-F:
5′-CGTTTGGGTTTCTTGGCT-3′
and
IFNG-R:
5′-TTTTTGTCACTCCTTTC-3′;
GZMB-F:
5′-GATTACGGGACATGAGG-3′
and
GZMB-R:
5′-TGACATTATGGAGCTCCCA-3′;
TNFA-F:
5′-GTAGCCCATTGTAGCAACC-3′
and
TNFA-R:
5′-TACTCTCAGCTCCACCA-3′.

### Example 5: In Vitro Killing Experiment of Axl-CAR-T Cells on Tumor Cells

This embodiment will use LCLC-103H with high expression of Axl HL60, MDA-MB-231, MDA-MB-453. MV4-11, And co-culture H460 cells with low expression of Axl and Axl-CAR-T cells obtained in Example 4 to investigate the killing ability of Axl-CAR-T cells against various tumor cells.

LCLC-103H and H460 are both human lung cancer cell lines, HL60 is a human leukemia tumor cell, and MV4-11 is a human acute monocytic leukemia cell line, all of which are derived from Nanjing Kebai Biotechnology Co., Ltd.

Both MDA-MB-231 and MDA-MB-453 are human breast cancer cells derived from ATCC.

Cultivate the above tumor cells separately in RPMI complete culture medium and passage them in a 1:3 ratio until corresponding logarithmic phase cells with good growth are obtained. Then resuspend each tumor cell in RPMI complete culture medium and adjust the concentration to obtain a suspension of each tumor cell. Take the T cells to be transfected from Example 4, resuspend them in RPMI complete culture medium and adjust the concentration to obtain a T cell suspension. Axl-CAR-T cells from Example 4 were resuspended in RPMI complete culture medium and the concentration was adjusted to obtain Axl-CAR-T cell suspension.

Take a 16 well plate pre coated with 50 microliters of RPMI complete culture medium per well, add tumor cell suspension to each well and adjust the concentration to 5000 cells per 100 microliters of culture medium before culturing until tumor cells adhere to the wall. Then: add T cell suspension to obtain a control group, and adjust the ratio of T cells to tumor cells (E:T); Add Axl-CAR-T cell suspension to obtain the experimental group and adjust E:T; add RPMI complete culture medium and adjust to the same amount as the control group and experimental group to obtain the blank group.

Real Time Cellular Analysis (RTCA) was used to monitor the blank group, control group, and experimental group corresponding to each tumor cell. Measure the cell index every 15 minutes and calculate the proliferation rate as (real-time cell index—cell index when T cells are added)/cell index when T cells are added.

The number of tumor cells in each well was adjusted to 3×10⁴ for the blank group, control group, and experimental group corresponding to each tumor cell. After 5 days of co-culture, the relative expression levels of IFNγ in each control group and experimental group compared to the blank group were detected using the Human IFNγ ELISPOT Kit (abcam).

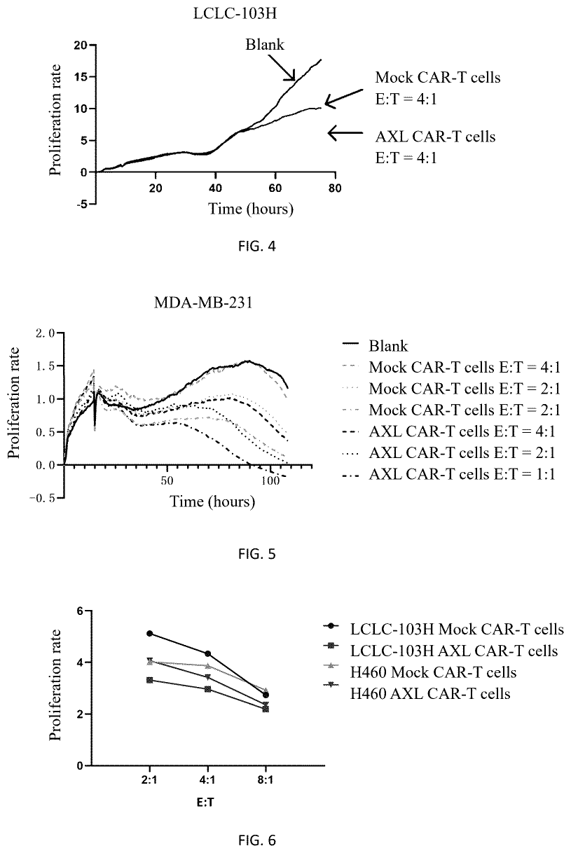

From the blank group of LCLC-103H shown in FIG. 4 and in E:T=4:1 The comparison chart of the changes in LCLC-103H proliferation rate between the control group and the experimental group at different co-culture times under condition 1 shows that, The killing effect of Axl-CAR-T cells on LCLC-103H is stronger than that of T cells in the control group, resulting in a significant decrease in the proliferation rate of LCLC-103H. It can be seen that Axl-CAR-T cells have a good killing effect on LCLC-103H.

From the comparison of the proliferation rates of MDA-MB-231 in the blank group, control group corresponding to different E:T ratios, and experimental group corresponding to different E:T ratios at different co-culture times shown in FIG. 5, it can be seen that under E:T ratios of 1:2 and 1:1, The killing effect of Axl-CAR-T cells on MDA-MB-231 was significantly better than that of the corresponding control group. It can be seen that Axl-CAR-T cells have a good killing effect on MDA-MB-231.

Axl-CAR-T cells have selective killing effects on tumor cells, as follows:

From the comparison of the proliferation rate trends of the corresponding control group and experimental group of LCLC-103H at different E:T shown in FIG. 6, as well as the comparison of the proliferation rate trends of the corresponding control group and experimental group of H460 at different E:T, it can be seen that Axl-CAR-T cells have good killing effects on LCLC-103H compared to the corresponding LCLC-103H control group at different E:T, at ratios of 2:1 and 4:1, compared to the corresponding H460 control group, the killing effect of Axl-CAR-T cells on H460 was significant. When E:T ratio was 8:1, compared to the corresponding H460 control group, the killing effect of Axl-CAR-T cells on H460 was not significant. Compared with the LCLC-103H group, the killing situation of each group showed strong dose dependence. E: At T ratios of 2:1 and 4:1, the killing effect of Axl CAR-T cells on LCLC-103H was more significant compared to their killing effect on H460, while at E:T ratios of 8:1, the killing effect of Axl CAR-T cells on LCLC-103H was not significant compared to their killing effect on H460.

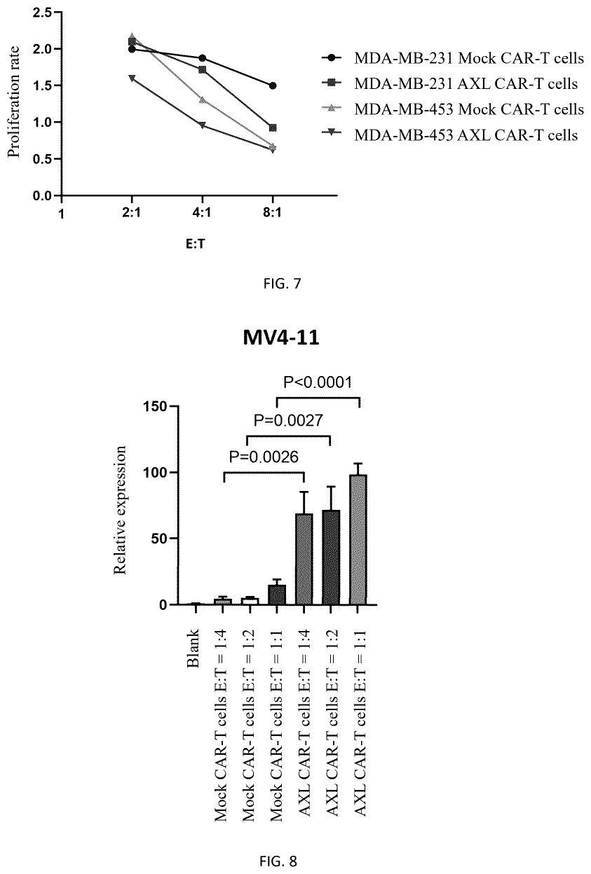

From the comparison of the proliferation rate trends of the corresponding control group and experimental group of MDA-MB-231 at different E:T shown in FIG. 7, as well as the comparison of the proliferation rate trends of the corresponding control group and experimental group of MDA-MB-453 at different E:T, it can be seen that when E:T is 4:1 and 8:1, Axl-CAR-T cells show a significant killing effect on MDA-MB-231 compared to the corresponding MDA-MB-231 control group, and when E:T is 2:1; the killing effect of Axl-CAR-T cells on MDA-MB-231 was not significant compared to the corresponding control group. At E:T ratios of 2:1 and 4:1, Axl-CAR-T cells showed significant cytotoxicity against MDA-MB-453 compared to the corresponding MDA-MB-453 control group, with E:T ratios of 2:1; the killing effect of Axl-CAR-T cells on MDA-MB-453 was not significant compared to the corresponding MDA-MB-453 control group. Under the same E:T conditions, Axl-CAR-T cells showed stronger killing performance against MDA-MB-453.

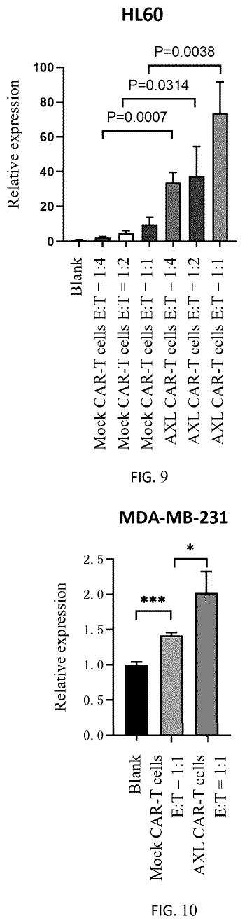

Due to the important immunomodulatory role of IFNγ as a cytokine secreted by immune active cells in inducing antiviral immunity, it is widely recognized in the industry that the level of IFNγ in the matrix after immune stimulation actually reflects the activity of effector cells. Therefore, detecting the level of IFNγ indirectly detects the activity of effector cells. The comparison of relative expression levels of IFNγ among the blank group, control group, and experimental group corresponding to MV4-11, HL60, and MDA-MB-231 shown in FIGS. 8 to 10 shows that compared with the blank group and control group corresponding to each tumor cell, the relative expression levels of IFNγ in each experimental group have a significant upward trend. Axl-CAR-T cells demonstrate good cytotoxicity against MV4-11, HL60, and MDA-MB-231.

### Example 6: In Vivo Killing Experiment of Axl-CAR-T Cells on Tumor Cells

Ten female NSG mice (Shanghai Nanfang Model Biotechnology Co., Ltd.) aged 6-8 weeks each were used for in vivo killing experiments. Specifically, 1×10⁷ MDA-MB-231 cells were implanted into the fourth mammary pad of each mouse, and tumor formation occurred after 18 days. Then intervention was performed to form the following PBS group, animal control group, and animal treatment group:
PBS group: Inject 100 microliters of PBS solution into the tail vein each time;
Animal control group: Resuspend the T cells to be transfected from Example 4 in 100 microliters of PBS solution and adjust the cell count to 8×10⁶. Administer tail vein injection to mice each time;
Animal treatment group: Resuspend Axl-CAR-T cells from Example 4 in 100 microliters of PBS solution and adjust the cell count to 8×10⁶. Administer tail vein injection to mice each time.

The above groups are injected via tail vein every two days, for a total of four injections. On the 34th day after the start of drug administration, euthanize the mice and collect tissues for analysis.

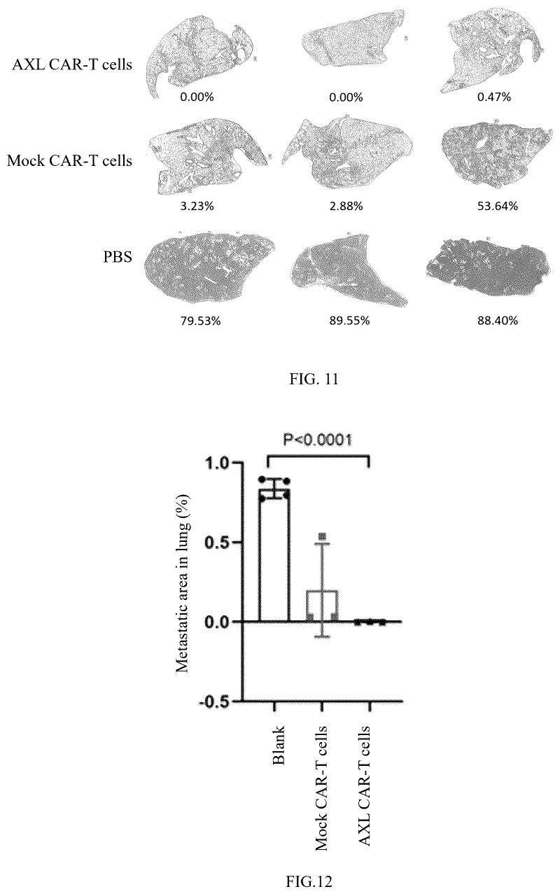

In this example, lung tissue samples from mice were soaked in 4% paraformaldehyde for fixation, then embedded in paraffin, sliced, and stained with hematoxylin eosin (HE) before microscopic examination. ImageJ was used for HE staining image processing, and the tumor part in the lung tissue was circled and the area was calculated. The comparative photos of tumor metastasis in each lung tissue sample of PBS group, animal control group, and animal treatment group shown in FIG. 11 were obtained, as well as the comparison of the proportion of lung metastasis areas in each group of lung tissue samples based on the photos shown in FIG. 11. The specific staining, microscopic examination, and image acquisition and analysis methods are routine technical means for technicians in this field. Referring to FIG. 11 and FIG. 12, compared with the PBS group and the animal control group, the proportion of lung tumor areas in the animal experimental group decreased significantly or even approached 0, which shows that Axl-CAR-T cells can inhibit the lung metastasis of breast cancer.

In this example, in situ tumor tissue samples were taken from each mouse to calculate tumor volume=0.5×L×W²; Where L is the length and W is the width. According to calculations, the average tumor volume of the PBS control group is 2800 cubic centimeters, the average tumor volume of the animal control group is 2430 cubic centimeters, and the average tumor volume of the animal experimental group is 1650 cubic centimeters. Relative to the control group, p=0.0005.

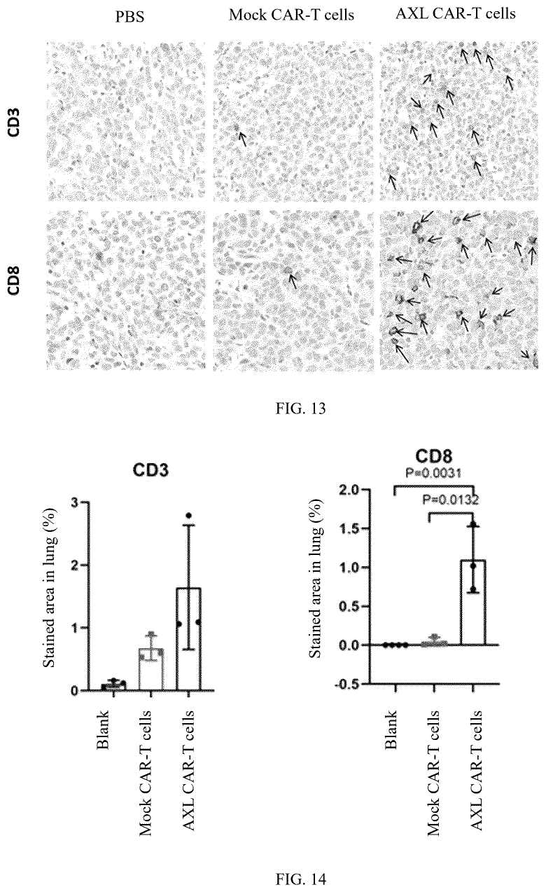

The in situ tumor tissue samples of each mouse were soaked in 4% paraformaldehyde for fixation, then embedded in paraffin, sliced, and subjected to immunohistochemical staining (IHC). The antibodies and ratios used included: anti-CD3 1:100 (Servicebio, GB13014); anti-CD8 1:100 (Servicebio, GB13068); Secondary antibody HRP goat anti rabbit antibody 1:200 (Servicebio, GB23303). Then, microscopic examination was performed, and ImageJ was used for immunohistochemical image processing. IHCTool was used to extract immunohistochemistry positive areas and calculate their area. The comparison of immunostaining photos of in situ tumor tissue samples in each group shown in FIG. 13, as well as the comparison of the proportion of CD3 and CD8 positive areas in the in situ tumor tissue samples in each group shown in FIG. 14, were obtained. Referring to FIGS. 13 and 14, compared to the animal control group, the protein levels of CD3 and CD8 were significantly upregulated in the tumor samples of the animal experimental group.

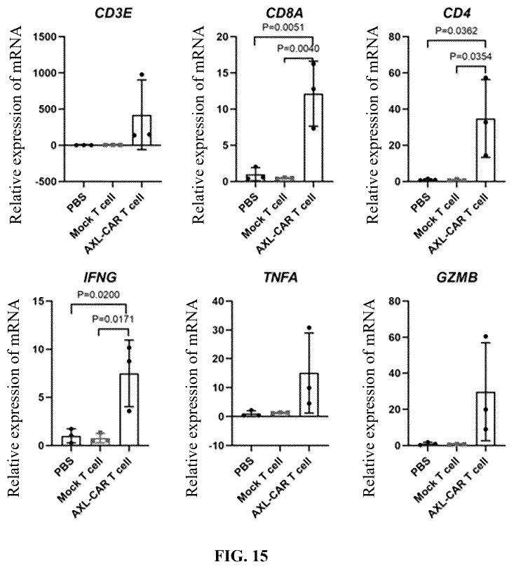

Furthermore, in this example, mRNA was extracted from various in situ tumor tissue samples for qPCR analysis, and the expression levels of CD3, CD8A, CD4, IFNG, GZMB, and TNFA were detected. The comparative expression levels of the above factors in each group were obtained as shown in FIG. 15. Among them, PBS represents the PBS group, Mock T cell represents the animal control group, and AXL-CAR T cell represents the animal experimental group. Please refer to the previous section for specific analysis steps. Referring to FIG. 15, compared to the animal control group, the tumor samples of the animal experimental group showed significant upregulation of the three T cell marker genes CD3, CD8A, and CD4. The cytokine genes IFNG, GZMB, and TNFA secreted by T cells were also significantly upregulated. Combined with the significantly upregulated protein levels of CD3 and CD8 in the tumor samples of the animal experimental group indicated in FIGS. 13 and 14, it was demonstrated that Axl-CAR-T cells have a stronger ability to infiltrate tumors compared to the animal control group.

What is claimed is:

1. A chimeric antigen receptor comprising a heavy chain variable region and a light chain variable region;
sequence of the light chain variable region is shown in SEQ ID NO: 1;
sequence of the heavy chain variable region is shown in SEQ ID NO: 2.

2. The chimeric antigen receptor according to claim 1, further comprising a connecting region connecting the light chain variable region and the heavy chain variable region, wherein sequence of the connecting region is shown in SEQ ID NO: 3.

3. The chimeric antigen receptor according to claim 1, further comprising a transmembrane domain and a hinge region located between the transmembrane domain and the heavy chain variable region of the Axl specific antibody;
sequence of the hinge region is derived from at least one of CD8, CD8 α, CD28, 4-1BB, ICOS, OX40, CD40, CD80, and IgG;
sequence of the transmembrane domain is derived from at least one of CD2, CD27, LFA-1, CD8 α, CD28, 4-1BB, ICOS, OX40, CD40, CD80, CD3 ζ, and CD3 ε.

4. The chimeric antigen receptor according to claim 3, wherein the sequence of the hinge region is shown in SEQ ID NO: 4, and the sequence of the transmembrane domain is shown in SEQ ID NO: 5.

5. The chimeric antigen receptor according to claim 3, further comprising an expression driver region and a signal peptide located between the expression driver region and the light chain variable region of the Axl specific antibody;
sequence of the expression driving region is derived from at least one of EF1 α, CMV, PGK, MPSV, MMLV, and SFFV.

6. The chimeric antigen receptor according to claim 5, wherein the sequence of the expression driving region is shown in SEQ ID NO: 6, and the sequence of the signal peptide is shown in SEQ ID NO: 7.

7. The chimeric antigen receptor according to claim 1, wherein the sequence of the chimeric antigen receptor is shown in SEQ ID NO: 8.

8. A nucleic acid characterized by encoding at least a partial fragment of the chimeric antigen receptor of claim 1.

9. A carrier comprising the nucleic acid of claim 8.

10. A host cell comprising the vector of claim 9.

11. An application of the chimeric antigen receptor of claim 1 in preparation of tumor therapeutic drugs or tumor therapy.


SEQUENCE LISTING (8 sequences)

SEQ ID NO: 1 - DNA, 318 bp, synthetic construct
  source 1..318: mol_type = other DNA
gagatcgtgctgacccagagccccgctacactgtctttaagccccggtgagagggccacattaagctgcagcgccagcagcagcatcggctacatgtactggtaccagcagaagcccggtcaagctcctcgtctgctgatctatttaaccagcaatttagccagcggcatccccgctagattcagcggcagcggaagcggcaccgactacactttaaccatcagctctttagagcccgaggacttcgccgtgtactactgccagcagtggagcagcaaccctcccaccttcggccaaggtaccaagctggagatcaag

SEQ ID NO: 2 - DNA, 348 bp, synthetic construct
  source 1..348: mol_type = other DNA
caagttcagctggtgcagtccggcgctgaggtgaagaagcccggtgcctccgtgaaggtgagctgcaaggccagcggctaccccttcaccgacttctacatcaactgggtgagacaagctcccggccaaggtttagagtggatgggctggatctaccccggctccggcaacaccaagtacaacgagaagttcaagggtcgtgtgaccctgactgtggacaccagcatcagcaccgcctacatggagctgtctcgtctgaggagcgacgacaccgccgtgtactactgcgctcgttccaccggcttcttcgactactggggccaaggtactttagtgacagtgagcagc

SEQ ID NO: 3 - DNA, 45 bp, synthetic construct
  source 1..45: mol_type = other DNA
ggtggaggtggcagcggaggaggtgggtccggcggtggaggaagc

SEQ ID NO: 4 - DNA, 135 bp, synthetic construct
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 5 - DNA, 539 bp, synthetic construct
  source 1..539: mol_type = other DNA
ttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 6 - DNA, 1182 bp, synthetic construct
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 63 bp, synthetic construct
  source 1..63: mol_type = other DNA
atggccttaccagtgaccgccttgctcctgccgctggccttgctgctccacgccgccaggccg

SEQ ID NO: 8 - DNA, 2652 bp, synthetic construct
  source 1..2652: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattcgaagccgccaccatggccttaccagtgaccgccttgctcctgccgctggccttgctgctccacgccgccaggccggagatcgtgctgacccagagccccgctacactgtctttaagccccggtgagagggccacattaagctgcagcgccagcagcagcatcggctacatgtactggtaccagcagaagcccggtcaagctcctcgtctgctgatctatttaaccagcaatttagccagcggcatccccgctagattcagcggcagcggaagcggcaccgactacactttaaccatcagctctttagagcccgaggacttcgccgtgtactactgccagcagtggagcagcaaccctcccaccttcggccaaggtaccaagctggagatcaagggtggaggtggcagcggaggaggtgggtccggcggtggaggaagccaagttcagctggtgcagtccggcgctgaggtgaagaagcccggtgcctccgtgaaggtgagctgcaaggccagcggctaccccttcaccgacttctacatcaactgggtgagacaagctcccggccaaggtttagagtggatgggctggatctaccccggctccggcaacaccaagtacaacgagaagttcaagggtcgtgtgaccctgactgtggacaccagcatcagcaccgcctacatggagctgtctcgtctgaggagcgacgacaccgccgtgtactactgcgctcgttccaccggcttcttcgactactggggccaaggtactttagtgacagtgagcagcggatccaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg